(19) United States
(12) Patent Application Publication
(10) Pub. No.: US 2006/0073534 A1
(43) Pub. Date: Apr. 6, 2006

(54) Method for cleaving and deglycosylating antibodies to promote ligand binding

(75) Inventors: Lisa Kelly, Athens, GA (US); J. David Puett, Athens, GA (US); J. Michael Pierce, Athens, GA (US)

Correspondence Address: KING & SPALDING LLP, 191 PEACHTREE STREET, N.E., 45TH FLOOR, ATLANTA, GA, 30303-1763, US

(73) Assignee: The University of Georgia Research Foundation, Inc, Atlanta, GA (US)

(21) Appl. No.: 10/958,566

(22) Filed: Oct. 5, 2004

(51) Int. Cl. G01N 33/537

(52) U.S. Cl. 435/7.92

(57) ABSTRACT

The present invention details methods to detect glycoproteins in samples to identify a disease state. In particular, an improved sandwich assay is described, using both an antibody and an array of lectins. Methods for increasing the efficiency of interaction of an antibody with a ligand by cleaving the antibody to remove the carbohydrate moiety, and orienting the cleaved fragment on a matrix to enhance recognition of a ligand are also described. Methods of differentiating pregnancy-derived and disease derived samples are also described.

### FIELD OF THE INVENTION

The present invention details methods to detect glycoproteins in samples to identify a disease state using binding assays and methods for increasing the efficiency of interaction of an antibody with a ligand by cleaving the antibody and orienting it on a matrix.

### BACKGROUND

Early diagnosis is one of the primary determinants of mortality and morbidity from disease. The recognition of a variety of diseases, notably cancers, in apparently healthy subjects and subsequent treatment thereof is still problematic. Alterations of post-translational modifications in readily attainable samples from patients are the best indicators of the disease state, although most potential markers are as yet unknown. Therefore there exists a need to identify new and more specific markers of disease.

A. Glycoproteins

The covalent attachment of oligosaccharides to protein is the most common post-translational event and occurs in more than 50% of proteins, independent of membrane linkage. Glycosylation occurs at specific locations along the polypeptide backbone of the protein. There are usually two major types of glycosylation: glycosylation characterized by O-linked oligosaccharides, which are attached to serine or threonine residues; and glycosylation characterized by N-linked oligosaccharides, which are attached to asparagine residues in an Asn-X-Ser/Thr sequence, where X can be any amino acid except proline. N-acetylneuramic acid (also known as sialic acid) is usually the terminal residue of both N-linked and O-linked oligosaccharides. Variables such as protein structure and cell type influence the number and nature of the carbohydrate units within the chains at different glycosylation sites. Glycosylation isomers are also common at the same site even within a given cell type. The levels of glycosylation are a reflection of the levels and activities of different glycosyltransferases and glycosidases responsible for the intracellular construction of oligosaccharides.

B. Glycosylation in Disease States

There is now overwhelming evidence that glycosylation of the glycoproteins produced by diseased cells is markedly altered. An increase in glycosylation is a common feature in the transformation to malignancy of certain cancers and has been related to the metastatic potential of tumor cell lines. Altered glycosylation of membrane glycoproteins and glycolipids is observed in mammalian cells transformed with diverse tumor viruses, carcinogens, or transfection with certain oncogenes. In some cases, there is a quantitative increase in a particular substituent, e.g., sialylation (Dall'Olio F. Protein glycosylation in cancer biology: an overview. Clin Mol Pathol 49:M126-M135 (1996)). In other instances, there is the reappearance of an oligosaccharide structure in the tumor which is normally only found in fetal tissue; for instance, certain Lewis histo-blood group antigens have been detected in adenocarcinomas. Qualitative differences in oligosaccharides may also be observed in certain transformed cells. For example, BHK fibroblasts transformed with polyoma virus or with Rous sarcoma virus display more highly branched complex N-linked oligosaccharides than do the corresponding normal cells. The increase in the β-(1,6) branching of the cell surface-bound oligosaccharides has been associated, at least in some cases, with capacity for metastasis. Increased levels of β-1,6 branching over the level in normal tissue has been observed for some human breast tumor tissues.

Studies have shown that increases in glycosylation have the potential to be used in diagnostic tools for disease states, in particular for cancers. A variety of carbohydrate markers have been used to reveal the importance of detailed oligosaccharide information to recognizing and characterizing carcinomas. Carbohydrate profiles of the primary tumor have been correlated with tumor grade, metastatic potential, and disease prognosis (Litynska, et al., Melanoma Res., 205-212 (2001); Hakomori, S., Adv. Cancer Res., 52:257-331 (1989)). Despite the fact that malignant alterations in sugar chains are quite variable, they are also reproducible. Therefore, comparison of the sugar chains of malignant cells and their normal counterparts provides an important diagnostic tool.

C. Human Chorionic Gonadotropin

Because cancer cells can have the characteristics of undifferentiated cells, one group of proteins that are a target of investigation are the oncofetal proteins(Coggin J H Jr. The implications of embryonic gene expression in neoplasia. Critical Reviews in Oncology-Hematology. 5(1):37-55, (1986)). One specific example of these is human chorionic gonadotropin (hCG), which is expressed by a variety of very common cancers (Triozzi P L. Stevens V C. Human chorionic gonadotropin as a target for cancer vaccines Oncology Reports. 6(1):7-17 (1999)). hCG is a 38 kDa heterodimeric glycoprotein hormone that is approximately 30% oligosaccharide by weight. Its 92 amino acid α-subunit is nearly identical to the α-subunits of human luteinizing (hLH), follicle stimulating (FSH), and thyroid-stimulating (TSH) hormones. hCG is structurally a member of the superfamily of ‘cystine-loop’ growth factors, along with transforming growth factor β (TGFβ), nerve growth factor (NGF), and platelet-derived growth factor β (PDGFβ), that are characterized by distinctive folds stabilized by disulfide bonds (Lapthorn et al., Nature, 369, 455-461 (1994)). Trophoblastic cells during pregnancy produce hCG, where it plays a central role in maintaining pregnancy by rescuing the corpus luteum. What has become more evident is that the pituitary and other normal tissues, such as bladder, prostate, and testis, can also produce small amounts of hCG (Bellet et al., Cancer Res., 57, 516-523 (1997); Hoermann et al., J. Clin. Endocrinol Metab, 71, 179-186 (1990); Lazar et al., Cancer Res., 55, 3735-3738 (1995)).

hCG occurs in a number of molecular forms in serum and urine, including intact hCG, free subunits, and the ‘β-core fragment’. During pregnancy, intact hCG is the main form in serum, although the β-core fragment is the major form in the urine and is believed to be derived from proteolysis in the kidneys. During pregnancy, the intact hCG heterodimer binds to an ovarian hLH/hCG receptor. Although expression of this receptor was initially considered to be restricted to gonadal tissues, recent studies have indicated low levels of expression on a variety of normal and neoplastic tissues, including those derived from breast and prostate (Lojun et al., Biol. Reprod., 57, 1202-1210 (1997); Dirnhofer et al., Prostate, 35, 212-220 (1998)).

The production of hCG by tumors arising from trophoblastic/germ cells, including choriocarcinoma, hydatidiform mole, and embryonal carcinoma of the testis, is an essential marker of the activity of these diseases, and its measurement plays a central role in their clinical management. That a wide variety of tumors of non-trophoblastic origin also express hCG is also well recognized. However, the spectrum of expression of hCG by non-trophoblastic cancers and its significance are somewhat controversial.

Braunstein reviewed hCG expression in 1990 and surmised that immunoreactive hCG could be demonstrated in the sera of approximately 18% of patients with non-trophoblastic malignancies (Braunstein, G D, Marcel Dekker, Inc., New York, 673-701 (1990)). hCG was detected most commonly in the sera of patients with uterine cervical (34%), ovarian (29%) pancreatic (27%), bladder (23%), hepatic (22%), and gastric (21%) cancers. Most of the studies reviewed, however, were based on the recognition of hCG, hCG-like material, or free hCG subunits by either polyclonal or monoclonal antibodies that were neither exceptionally specific nor of high affinity. Recently, more sensitive monoclonal-based assays highly specific for intact hCG, α-hCG, β-hCG, and the β-core fragment have enabled investigators to re-evaluate the production of hCG. For example, elevated serum hCG in 72% of patients with pancreatic cancer and in 9% of those with non-malignant disease has been found using a highly sensitive time-resolved immunofluorometric assays (Alfthan et al., Cancer Res., 52, 4628-4633 (1992)). Low-level hCG elevations and free α-hCG subunit, as measured by an immunoradiometric assay, has been reported to be common (Marcillac et al., Cancer Res., 52, 3901-3907 (1992)). Free β-hCG (≧100 pg/ml), however, was more tumor specific and was detected in 47% of bladder, 32% of pancreatic, and 30% of cervical cancer patients, in addition to a majority of patients with germ cell tumors. All normal subjects and disease controls had free β-hCG levels less than 100 pg/ml. Thus, a test for increased levels of hCG or β-hCG was thought to potentially allow detection of disease states. However, as described above, increased levels of hCG or β-hCG are also found during pregnancy, and have also been identified in a subset of normal individuals.

Although hCG is overexpressed in neoplastic tissues, there is no evidence at present that the hCG genes in neoplastic cells are mutated. There is, however, evidence that the glycosylation of hCG secreted by normal and malignant cells differs, with malignant cells producing hyper-branched N— and O-linked oligosaccharides (Elliott et al., Endocrine, 7, 15-32 (1997)). The same is true for the expression of other oncofetal antigens, such as carcinoembryonic antigen (CEA), α-fetoprotein, and members of the recently described MAGE/GAGE/BAGE family (Van den Eynde et al., Int. J. Clin. Lab. Res., 27, 81-86 (1997)).

Assays for hCG and its subunits are poorly standardized due to various factors, for example cross-reaction with related substances. Cross-reactions with LH can be a problem with assays based on polyclonal antibodies, though this can be eliminated with the use of monoclonal or a combination mono- and poly-clonal antibodies. Certain antibodies also do not recognize nicked hCG and β-hCG, causing considerable variations between assays (Cole et al., Clin. Chem., 38, 263-270 (1992)). Most commercial hCG assays used today are so called ‘total hCG’ assays because they detect both hCG and β-hCG. Similarly, designing assays for β-hCG is complicated by the fact that pregnancy serum samples contain vast (100-fold) excesses of hCG, which may interfere with these antibodies (Alfthan et al., Clin. Chem. (NY), 34, 1758-1762 (1988)).

D. Glycoprotein Detection

Most investigators use lectins to assess the presence of specific carbohydrates in a sample. Lectins are proteins with the ability to bind specific sugars. General reviews of lectins can be found in Lis and Sharon, “Lectins: Their Chemistry and Application to Immunology”, in The Antigens, Vol. IV, chap. 7, pp. 429-529 (Sela, ed.; Academic Press, N.Y.; 1977); Sharon and Lis, “Lectins: Cell-Agglutinating and Sugar-Specific Proteins”, Science, 177: 949-959 (1972); Lis and Sharon, “Lectins in Higher Plants”, in The Biochemistry of Plants, Vol. 6, Chap. 10, pp. 371-447 (Marcus, ed.; Academic Press, N.Y., 1981); Lis and Sharon, “The Biochemistry of Plant Lectins (Phytohemagglutinins)”, Ann. Rev. Biochem., 42: 541-574 (1973); Horowitz, et al., “Immunological Aspects and Lectins”, in The Glycoconjugates, Vol. 2, Chap. 7, pp. 387-449 (Horowitz and Pigman, eds.; Academic Press, N.Y.; 1978).

Several researchers have proposed using lectins in diagnostic assay because of their capacity to recognize the specific carbohydrate epitopes that can be associated with disease states. The principal technique used to analyze carbohydrate modification of proteins in native samples is by developing “glycosylation profiles” using lectin binding assays. These assays are usually performed by separating a cell or tissue extract and probing this using one or more conjugated lectin. The levels of lectin binding are thereafter compared between samples to identify protein bands with altered levels of glycosylation. For example, P.C.T Publication No. WO 89/04490 (Sumar et al. published May 18, 1989) discloses the use of multiple lectins to analyze the carbohydrate glycosylation pattern on IgG that has been isolated by known means from a serum samples in order to diagnose the presence of breast cancer. The lectins used include Abrus precatorius, Ricinus communis, Bandeiraea simplicifolia, Datura stramonium, and Lycoperiscon esculentum. Additional techniques to identify glycoprotein variations include serial lectin-affinity chromatography (Endo, 1996, J. of Chromatography. A 720(1-2):251-61). Sequential chromatography steps using different lectins are used to purify a subset of glycoproteins that can subsequently be identified by mass spectroscopy or protein sequencing. These techniques, because they rely only on glycoprotein binding to lectins, produce limited information and are not readily applied to the investigation of glycosylation of specific known proteins.

To investigate the glycosylation of a protein of interest, some method must be included in the analysis to specify this protein. The best known means to do this are by recognizing specific epitopes on a protein using one or more antibodies. The use of both antibodies and lectins in combination for the purpose of identifying specific glycoproteins was described, for example, in Kinoshita, et al., Clin. Chim Acta 179:143-152 (1989). In these assays, serum samples were sequentially incubated with an anti-α-fetoprotein polyclonal antibody and then with a peroxidase-labeled lectin for visualization.

Canfield (WO 87/00289, published Jan. 15, 1987) proposes a method of detecting soluble desialylated glycoproteins (particularly hCG, thyroglobulin, CEA and CA19-9) in biological fluids. His assay preferably uses a solid phase lectin and a labeled antibody, but he recognizes that the roles of the lectin and the antibody may be reversed.

David et al., (U.S. Pat. No. 4,376,110), disclose sandwich immunoassay techniques for determination of the presence and/or concentration of antigenic substances in fluids using monoclonal antibodies. One monoclonal antibody is presented in a soluble labeled form while a second monoclonal antibody is bound to a solid carrier. David teaches that it is important that the monoclonal antibody have a high affinity for the antigen, preferably at least 10⁸ liters/mole, and more preferably, at least 10⁹ liters/mole. David does not recognize that lectins may replace antibodies in a sandwich assay, and indeed teaches against such a substitution in view of the relatively low affinity of lectins for their targets.

Samuel et al. (U.S. Pat. No. 5,242,799) discloses a heterologous sandwich immunoassay for human TF antigen using a monoclonal antibody to capture the antigen from a sample of bodily fluid and a labeled lectin (peanut agglutinin) as a probe to confirm that TF antigen was captured.

Holtzhauer et al. (WO 99/39209, published Aug. 5, 1999) discloses a sandwich immunoassay for detecting the presence of fucosylated α-fetoprotein, using a specific antibody in conjunction with specific lectins (selected from the group of Ulex-europaeus agglutinin, Lotus-tetragonolobus agglutinin, and Anguilla-anguilla agglutinin). In addition, the use of covalent lectin-antibody conjugates in immunoassays is described in Chu, U.S. Pat. No. 4,371,515 (1983); Chu, U.S. Pat. No. 4,259,747; and Guesdon et al., J. Immunol. Meth., 39:1-13 (1980).

However, antibodies contain 3-12% carbohydrates at conserved N-glycosylation sites located in the constant regions of their heavy chains. Although glycosylation of this site seems to have little effect on antigen binding (Donadel, G., Calabro, A., Sigounas, G., Hascall, V. C., Notkins, A. L. and Harindranath, N. (1994) Glycobiology 4, 491-496), this glycan moiety provides a high level of background in assays which are designed to detect glycans. These assays do not account for the glycosylation of the antibodies themselves, lending unknown and variable background to each invention and limiting the benefit that can be gained by using them.

E. Antibody Deglycosylation

One method to overcome this high background is to deglycosylate the antibody. There are several known methods to deglycosylate antibodies, including both enzymatic and chemical procedures. However, these methods are both time consuming and could affect the activity as well as the stability of the antibody, particularly since the presence of a detergent seems to relate to the success of the procedure (Hobbs S M., et al. Molecular Immunology. 29(7-8):949-56, 1992). An example of this is disclosed by Kottgen, et al., (Biol. Chem. Hoppe-Seyler, 369:1157-1166 (October 1988), which describes an assay for glycosylated fibrinogen employing a deglycosylated solid phase anti-fibrinogen polyclonal antibody and a peroxidase-labeled lectin. The analyte was first incubated with the solid phase antibody, then a wash step removed unbound accompanying glycoproteins which would later compete for lectin binding. However methods to deglycosylate antibodies are time consuming and the potentially harsh chemical treatment could negatively affect the antibody binding to the ligand. In addition, because standard techniques to link antibodies to solid supports do not specify directionality, the apparent affinity of the antibody for the antigen can be greatly reduced.

F. Antibody Cleavage

Antibodies are comprised of four subunits: two light chains and two heavy chains, which associate via disulfide bonds and non-covalent interactions. Together the four subunits form a Y-shaped molecule, with the top of the “Y” being made of both heavy and light chains while the stem of the “Y” is a heavy chain dimer. Each chain is organized into a constant region, which differs only minimally between antibodies, and a variable region which comprises the antigen binding sites and is found at the amino-terminal of each chain, at the top of the “Y”. The sequence of the variable region determines the affinity of antibodies for specific antigens, which can differ dramatically. In addition, the method of production of an antibody drastically affects the utility of the antibody as a diagnostic tool. For example, the affinity of monoclonal antibodies is often lower than that of corresponding polyclonal antibodies (see for e.g. Chappey O. N., Pharm Res. 9(11):1375-9 (1992)).

Antibody fragments, as long as they contain variable regions, are effective in binding antigen. Many studies have shown that antibody fragments produced by recombinant means (i.e. by producing isolated fragments from recombinant DNA) can be used to detect antigens in samples and can be utilized to detect antigens by linking the fragment to a matrix (described in: Oelschlaeger, et al. Analytical Biochemistry. 309(1):27 (2002); Ramirez N., et al. Transgenic Research. 11(1):61-4 (2002); Hock B. , et al. Biosensors & Bioelectronics. 17(3):239-49 (2002); Foy B D., et al. Journal of Immunological Methods. 261(1-2):73-83 (2002); Battersby J E., et al. Journal of Chromatography. A. 927(1-2):61-76 (2001); Stausbol-Gron B., et al. European Journal of Biochemistry. 268(10):3099-107 (2001); Schmiedl A., et al. Protein Engineering. 13(10):725-34 (2000); Schmiedl A., et al. Journal of Immunological Methods. 242(1-2):101-14 (2000); Hom I R., et al. FEBS Letters. 463(1-2):115-20 (1999); Grant S D., et al. Journal of Agricultural & Food Chemistry. 47(1):340-5 (1999); Sanchez L., et al. Journal of Biotechnology. 72(1-2):13-20 (1999); Casalvilla R., et al. Journal of Biotechnology. 72(1-2):1-12 (1999); Hengerer A., et al. Biotechniques. 26(5):956-60, 962, 964 (1999); Reinhold U., et al. Journal of Investigative Dermatology. 112(5):744-50 (1999); Piervincenzi R T., et al. Biosensors & Bioelectronics. 13(3-4):305-12 (1998); Gill A., et al. Journal of Biotechnology. 48(1-2):117-27 (1996); De Jonge, et al. Molecular Immunology. 32(17-18):1405-12 (1995); Canaan-Haden L., et al. Biotechniques. 19(4):606-8, 610, 612 passim (1995); Graham B M., et al. Journal of Chemical Technology & Biotechnology. 63(3):279-89 (1995); Ayala M., et al. Biotechniques. 18(5):832, 835-8, 840-2 (1995); U.S. Pat. Nos. 5,648,237, 5,965,456; 5,648,237). Because recombinant antibodies must be developed for the success of these techniques, they are limited in that traditional methods of antibody development will not apply, nor can they be used on previously known and characterized antibodies. Because antibodies are difficult to make and do not always recognize native epitopes, these methods are all limited in the applications that they can be used in. There is also no suggestion in these references that deglycosylation is a purpose of the development of the antibody fragments, nor are there any further indications to utilize these fragments in conjunction with a glycan recognition agent.

Antibody fragmentation by enzymes has long been known in the art. The enzymes known include pepsin, papain, and ficin, which each release an Fc region (which comprises the constant region of the immunoglobulin and is in some cases fragmented), and either two F(ab) fragments (antigen binding) or one F(ab)₂ fragment comprised of the two individual F(ab) fragments and a short “hinge region” at its carboxy-terminal. Prior art references have shown that cleaved antibody fragments do not exhibit all the same properties as antibody fragments developed by recombinant means (see for e.g. U.S. Pat. No. 4,937,183), there is therefore reason to believe that utilizing known antibodies which have been cleaved will improve upon the use of recombinantly produced antibodies.

Standard procedures of linking antibodies or antibody fragments to a matrix often utilize the glycan moiety on the antibody for the linkage (see for e.g. U.S. Pat. Nos. 4,937,183 and 5,635,603). Additional references disclose linkage of antibody fragments to matrixes that are soluble, which reduces the utility of these matrices for diagnostic purposes (see e.g. U.S. Pat. No. 6,410,020; PCT Publication No. WO 98/25971). U.S. Pat. Nos. 6,365,418, 6,329,209, 6,329,209, and 6,406,921 all disclose the linkage of antibodies or antibody fragments to a specific solid support surface by, for example, disulfide linkage. However, these references all limit the surfaces to which the antibody fragments could be linked and do not suggest that deglycosylation should or would occur through any method. There is also no suggestion that one could combine this technique with a further step to identify multiple aspects of the antigen other than by adding multiple antibodies to make “patches” on a recognition array. U.S. Pat. No. 5,622,872 discloses antibody fragments bound to an electric sensor, wherein binding of an analyte can change the electrical, optical, or structural properties of a polymeric layer. This tool provides for a sensitive measurement, but only fluorometric changes are analyzed. There is therefore still no suggestion in the prior art that one can combine these teachings to limit lectin binding and enhance antibody recognition by cleaving known antibodies to yield antibody fragments and orient them on a matrix to allow enhanced recognition of analyte.

Therefore, despite these advances, a need continues to exist for a method with improved sensitivity, improved range and less manipulation to more rapidly analyze samples for the presence of a target glycoprotein. There is additionally a need to develop diagnostic tools to efficiently screen individuals for diseases such as cancer and potentially develop therapeutic devices by enhanced glycoprotein detection capacity of cleaved antibody and lectin combinations.

### OBJECT

Accordingly, it is an object of the present invention to provide a method for enhancing the sensitivity of assays to detect glycoprotein analytes in a solution.

It is a further object of the present invention to provide a method to enhance the sensitivity of assays designed to detect markers of disease.

Another object of the present invention is to provide methods for identifying glycoproteins that can act as disease markers because they possess glycosylation patterns that differ from the glycoproteins from non-diseased cells.

A further object of the present invention is to provide a diagnostic tool for detecting diseases from patient samples.

A specific object of the present invention to provide novel methods for the diagnosis and prognosis of cancers associated with elevated levels of human chorionic gonadotropin, or altered glycosylation patterns of hCG.

It is another object of the present invention to develop novel diagnostic assays for hCG, and altered glycosylation patterns on hCG associated with cancerous cells.

It is another object of the present invention to provide novel probes for detecting altered hCG glycosylation patterns on hCG molecules excreted from cancerous cells.

Yet another object of the present invention is to provide methods for identifying glycoproteins that can act as disease markers because they are differentially expressed in diseased versus non-diseased cells.

A further object of the present invention is to develop therapeutic devices which can target a cytotoxic agent to a diseased cell by virtue of the enhanced detection and reduced background afforded by a combination of lectin and antibody fragment recognition.

### SUMMARY

These and other objects are achieved by a method of enhancing the detection of unique glycosylation features of a protein associated with a disease by utilizing a heterologous sandwich type assay.

In a general embodiment of the invention, a capturing agent is used to recover a protein from a biological sample and a probe is used to recognize the carbohydrate moieties on the protein.

In one manifestation of the invention, the glycosylation pattern of a protein is analyzed by contacting a biological fluid with an antibody specific for the protein, forming a bound antibody-protein complex; contacting the bound protein with a carbohydrate probe; and detecting association between the bound protein and the carbohydrate probe. This aspect of the invention is most applicable to highly expressed proteins such as tumoral hCG.

In one embodiment of the invention, the capturing agent is an antibody specific for hCG and the probe is specific for the unique glycosylation pattern on tumoral hCG.

The increased sensitivity of this method relies in part on methods to improve the binding capacity on a matrix and reduce carbohydrate load of antibodies by cleaving these antibodies using an enzyme and linking the cleaved antibodies to a substrate, prior to analyte binding.

The inventors unexpectedly discovered that cleavage of an antibody and oriented linkage to a solid matrix increases the apparent antigen binding capacity of the antibody matrix. Therefore, one embodiment provides a method to improve the binding capacity of antibodies on a matrix by cleaving the antibodies with a protease and linking them in an oriented manner to a solid matrix. Because the antibodies are in this way aligned with their variable regions equally available for antigen binding, this method allows enhanced interaction with antigen and increased antibody load on the matrix. The invention utilizes antibodies prepared through traditional means, thereby expanding the utility of the invention beyond that of recombinant antibody fragments.

Another embodiment of this invention provides a higher affinity method to detect specific glycoproteins comprising: providing an antibody; cleaving said antibody using a protease to yield a cleaved antigen recognition fragment; linking said cleaved antigen recognition fragment to a solid matrix; contacting said cleaved antigen recognition fragment with an antigen, yielding a bound antigen; contacting said bound antigen with a carbohydrate-binding molecule; and recognizing the contacted carbohydrate-binding molecule. This method achieves a higher apparent affinity of the antibody for the antigen by orienting the antibody binding fragments (F(ab)) in a directional manner, as well as decreasing non-specific background binding of the carbohydrate-binding molecule when the cleavage renders the antibody free of carbohydrates, preferably N-linked oligosaccharides.

One aspect of this invention applies to the diagnosis of disease in tissue, urine, or serum samples. In particular the invention allows the analysis of glycoproteins found in diseased versus normal states. The inventors unexpectedly discovered that a sandwich-type assay that relies upon the unique glycosylation patterns of tumoral hCG can be used to distinguish tumoral hCG from pregnancy-associated hCG and other non-tumoral hCG. The carbohydrate probe in this instance preferably is specific for the presence of enhanced fucosylation and/or triantennary branching, or decreased terminal mannose, and most preferably is the Aleuria aurantia lectin, the Galanthus nivalis agglutinin, Sambucis nigra agglutinin, or Phaseolus vulgaris erythroagglutinin. Although the sandwich type-assay is the preferred vehicle for practicing the instant invention, it will be appreciated by workers of skill in the art that other assays that employ this dual targeting scheme can also be constructed.

This discovery gives rise to a number of uses in the prevention and treatment of benign, malignant, metastatic, and non-metastatic cancers, including:
Determining the presence of a carcinoma initially;
Distinguishing tumoral hCG from pregnancy-associated hCG;
Staging the carcinoma and thus providing guidance for future treatment (systemic versus localized treatment);
Following a course of treatment of a carcinoma; and
Determining recurrence of a carcinoma

The dual targeting feature of the instant invention substantially reduces the possibility of a false positive signal because a positive reactivity in this assay depends on the ability of the antigen to meet the epitope requirements of two different anti-hCG reagents, (e.g., a capture antibody and a carbohydrate probe).

### DETAILED DESCRIPTION

Definitions

Human choriogonadotropin: Human choriogonadotropin, sometimes abbreviated “hCG,” is defined as the intact heterodimer consisting of an α- and a β-subunit.

Free β subunit: The β subunit of hCG refers to the non-combined β-subunit consisting of a 23 kD polypeptide containing 145 amino acid residues. The β-subunit contains 2 N-glycosidic moieties attached to asparagine residues 13 and 30, a carboxyl terminal extension peptide (CTP), and four O-glycosidic linked oligosaccharides bound to serine residues 121, 127, 132, and 138.

Free α subunit: The α subunit of hCG refers to the non-combined α-subunit consisting of a glycosylated 14 kD polypeptide containing 92 amino acid residues and two N-linked complex branched oligosaccharide moieties bound to asparagine residues 52 and 78. It is common to hCG, LH, FSH and TSH.

Core fragment of hCGβ: The core fragment of hCGβ refers to a 10 kD two-chain polypeptide lacking the amino acid sequences 1-5, 41-54 and 93-145. The two chains are held together by disulfide bonds. The term may be abbreviated hCGβcf.

Aleuria aurantia lectin: The Aleuria aurantia lectin is a dimer of two identical subunits of about 36,000 daltons each with an isoelectric point of about pH 9, isolated from Aleuria aurantia mushrooms. Unlike Ulex europaeus and Lotus tetragonolobus lectins which prefer (α-1,2) linked fucose residues, Aleuria aurantia lectin binds preferentially to fucose linked (α-1,6) to N-acetylglucosamine or to fucose linked (α-1,3) to N-acetyllactosamine related structures.

Antibody load (on a matrix): In this application this is defined as the number of antibody molecules that can be accommodated on a given area of a binding matrix.

Phaseolus vulgaris erthroagglutinin: Phaseolus vulgaris erythroagglutinin is a member of the Phaseolus vulgaris agglutinin family of lectins, each of which consists of four subunits, isolated from Phaseolus vulgaris (red kidney bean) seeds. The Phaseolus vulgaris erythroagglutinin appears to be involved primarily in red cell agglutination and has been designated the “E” subunit (for erythroagglutinin). This lectin appears to have a high specificity for bi- and/or tri-antennary bisected complex N-linked sequences.

Galanthus nivalis agglutinin (GNA): GNA is a snowdrop lectin. Part of a family of lectins that also include Daffodil (Narcissus pseudonarcissus agglutinin, NPA) and are usually thought to recognize mannose residues. Additionally, they demonstrated a more complex specificity, than for example Con A, in that they distinguished Man-Man linkages. GNA recognizes terminal mannose, α(1-3), α(1-6) or α(1-2) linked to mannose; thus it is suitable for identifying “high mannose” N-glycan chains or O-glycosidically linked mannoses in yeast glycoproteins

Sambucis nigra agglutinin (SNA): From the elderberry. Recognizes sialic acid linked α(2-6) to galactose; thus it is suitable for identifying complex, sialylated N-glycan chains in combination with the lectin MAA; correspondingly linked sialic acids in O-glycan structures are also recognized.

Tumoral hCG: The term tumoral hCG refers to hCG derived from cancer cells that express abnormal levels of hCG or abnormally glycosylated hCG when compared to hCG excreted as a consequence of pregnancy, or baseline hCG expression from non-cancerous cells.

Cancer: Cancer is meant to include pre-malignant conditions, malignant cancer, metastatic cancer, and non-metastasized cancer. Such cancers include trophoblastic cancers such as choriocarcinoma, hydatidiform mole, and embryonal carcinoma of the testis, as well as non-trophoblastic cancers such as uterine cervical cancer, ovarian cancer, pancreatic cancer, lung cancer, prostate cancer, breast cancer, bladder cancer, hepatic cancer, and gastric cancer. The invention has particular utility in diagnosing or treating diseases associated with altered levels of hCG, such as cancers that secrete hCG or its subunits.

Antibody Apparent Affinity

In one broad aspect, the invention provides methods to enhance the efficacy of binding between a known antibody and an antigen, thus increasing the apparent affinity of the antibody, by cleaving the antibody and linking it in an oriented manner to a surface. Although not to be held by theory, this is thought to be due to a higher antibody load found on the matrix. Thus, in one embodiment, the invention is towards increasing the antibody load on a given matrix. The antibodies of the invention can be any known antibodies, from any class. The antibodies can include polyclonal and monoclonal antibodies, as well as antigen binding fragments of such antibodies. Methods of preparing polyclonal or monoclonal antibodies are well known to those skilled in the art (see, for example, Harlow and Lane, Antibodies: A Laboratory Manual (1988)). An antibody useful in the invention, or antigen binding fragment of such an antibody, is characterized by having specific binding activity for a ligand or sample epitope. Specific binding activity of an antibody for a ligand can be readily determined by one skilled in the art, for example, by comparing the binding activity of an antibody to a particular ligand versus a control ligand that differs from the particular ligand. Both naturally occurring as well as non-naturally occurring antibodies may be utilized in this invention, including, for example, chimeric, bifunctional and humanized antibodies. Non-naturally occurring antibodies can be constructed using solid phase peptide synthesis, can be produced recombinantly or can be obtained, for example, by screening combinatorial libraries consisting of variable heavy chains and variable light chains as described by Huse et al. (Science 246:1275-1281 (1989); Kang et al., Proc. Natl. Acad Sci. USA, 88:4363-4366 (1991)). The advantage of using such a combinatorial antibody library is that antibodies do not have to be individually generated for each ligand. These and other methods of making functional antibodies are well known to those skilled in the art (Winter and Harris, Immunol. Today 14:243-246 (1993); Ward et al., Nature 341:544-546 (1989); Hilyard et al., 1992, Protein Engineering: A practical approach; Borrabeck, 1995, Antibody Engineering, 2d ed.).

Specific Antibodies

In one preferred embodiment of the present invention, the antibody recognizes an epitope on hCG. Polyclonal or monoclonal antibodies specific for hCG can be prepared by using hCG as an immunogen to challenge a mammal, e.g. a mouse, rabbit, rat or goat. The antibody can be raised against the entire hCG molecule, the hCGα subunit, the hCGβ subunit, hCGβcf, or any immunogenic determinant thereof. The antibody is most preferably specific for the β-subunit of the human chorionic gonadotropin molecule. Those skilled in the art will recognize that various protocols may be used. Preferably, the animal is challenged over a substantial period (e.g. 5 months). See generally, Haavik et al., Glycobiology 2:217-224 (1992). For mouse monoclonal antibodies, standard fusion partners may be used to immortalize spleen cells from the challenged animal. See, e.g., Kohler and Milstein, Nature 256:495-497 (1977). The resulting anti-hCG monoclonal antibodies or polyclonal antibodies may be used in the various immunological procedures described below.

Antibody Fragments

After the antibody has been generated, and before it is immobilized on a carrier for use in practice of the instant invention, the antibody is preferably treated to remove any sugars on the antibody that might inadvertently compete for binding by the carbohydrate probe. Techniques for cleaving carbohydrates from glycoproteins can include treatment with a deglycosylating enzyme such as N-glycanase.

In the most preferable embodiment of the invention, the antibody is cleaved by a protease before contacting it with an antigen. This embodiment is preferable because the inventors have surprisingly shown that this can increase the apparent affinity of the immobilized antibody for an antigen. The protease can be any protease that releases a fragment at least containing the variable antibody recognition region of the antibody. Suitable protease enzymes include, but are not limited to: papain, chymopapain, bromelain, ficin, tryspin, etc. Further proteases that may be used in the present invention can be found at http://merops.sanger.ac.uk/index.html. The thiol protease enzymes are activated by reaction with cysteine or a low molecular weight sulfhydryl reducing agent such as dithiothreitol, mercaptoethanol, mercaptoethylamine, etc. In practice, an antibody is reacted with an amount of an activated thiol protease enzyme equivalent (on a weight basis) to about 1-20% of the antibody composition. The reaction mixture may be buffered using a buffer such as phosphate, citrate or acetate buffer at a pH from about 5.0-7.0, preferably pH 5.0-6.0. The reaction mixture is incubated at a temperature from about 4° C.-37° C., generally about room temperature (about 25° C.). The reaction is preferably performed in the presence of a chelating agent such as, for example, ethylenediaminetetraacetic acid (EDTA) at about 1-3 mM, which enhances the stability of the thiol protease enzyme. In practice, the thiol protease enzyme is preactivated by incubation with cysteine or a sulfhydryl reducing agent such as dithiothreitol, mercaptoethanol, mercaptoethylamine, etc. For example, a thiol protease enzyme may be activated by dissolving the enzyme at about 1 mg/ml in 50 mM cysteine-containing buffer and incubating the reaction mixture for about 1 hour at room temperature. The cysteine can be removed by gel filtration chromatography (“desalting”) and then the eluted activated enzyme can be used as described to digest an antibody composition. For convenience and ease of separation of the antibody binding fragment (F(ab)) compositions formed, the enzyme can be immobilized by attachment to a substrate such as agarose, Sepharose. Sephadex, polyacrylamide, or agarose-acrylamide beads, before activation. For example, agarose beads activated by reaction with carbonyldiimidazole (commercially available as “Reacti Gel” Pierce Chemical Corporation) can be used to immobilize the thiol protease enzyme employed in the present reaction scheme. Further, the antibody fragment can be separated by gently reducing interchain disulfide bonds with such reagents as mercaptoethanolamine.

In this application, the term “antibody fragment” refers to any derivative of an antibody which is less than full-length. Preferably, the antibody fragment retains at least a significant portion of the full-length antibody's specific binding ability. Examples of antibody fragments include, but are not limited to, Fab, Fab′, F(ab′)2, scFv, Fv, dsFv diabody, and Fd fragments. The antibody fragment may optionally be a single chain antibody fragment. Alternatively, the fragment may comprise multiple chains which are linked together, for instance, by disulfide linkages. The fragment may also optionally be a multimolecular complex. Single-chain Fvs (scFvs) are antibody fragments consisting of only the variable light chain and variable heavy chain covalently connected to one another by a polypeptide linker. Either the variable light chain or the variable heavy chain may be the amino-terminal domain. The polypeptide linker may be of variable length and composition so long as the two variable domains are bridged without serious steric interference.

The antigen binding domains can be separated from other cleaved products by a variety of methods. In one embodiment, the Fc domain is removed by incubation with a protein A or G (Staphylococcus aureus) linked to a support that can be separated (these are well known in the art and many versions are commercially available).

Linking Antibody to Support

An aspect of this invention is to link the antibody binding fragments to a solid support in an oriented manner. This can be accomplished by a variety of methods. In a preferred embodiment, the antibody fragment is linked to a solid support via a thiol linkage based on a cysteine residue in the antibody “hinge” region. However, it will be appreciated that other methods of orienting the antibody are also applicable. For example, the antibody can be linked to a solid support via a carboxy-terminal amino acid other than a cysteine via, for example, an amide linkage. Additionally, the antibody fragment can be linked to a linking agent, such as streptavadin, and thereafter linked to a biotinylated surface by interaction of the streptavidin and biotin.

Solid supports which may be employed include, but are not limited to, those made of cellulosic materials such as paper, cellulose, and cellulose derivatives such as cellulose acetate and nitrocellulose; silica; fiberglass; inorganic materials such as deactivated aluminum, diatomaceous earth, or other inorganic finely divided material dispersed uniformly in a porous polymer matrix made of polymers such as vinyl chloride-propylene copolymer and vinyl chloride-vinyl acetate copolymer; cotton; nylon; porous gels such as silica gel, agarose, dextran, and gelatin; polymeric films such as polyacrylamide; magnetic particles; microtiter plates; polystyrene tubes; protein-binding membranes; Sephadex (Pharmacia Fine Chemicals, Inc., Piscataway, N.J.); Trisacryl (Pointet-Girard); silicon particles; polystyrene particles; vinyl chloride particles; latex particles; and porous fibrous matrices.

Detection of hCG Glycosylation

As the inventors have unexpectedly discovered that tumoral hCG can be distinguished from non-tumoral hCG by the presence of enhanced fucosylation or branching of carbohydrate moieties. Thus, in one specific embodiment of the invention, hCG is separated from a complex solution (such as human urine) for analysis of its glycan moieties. Isolation of hCG may be performed by numerous methods which are well-known to those of the skill in the art. (See Sambrook, et al., Molecular Cloning; A Laboratory Manual, 2nd ed., Cold Spring Harbor, N.Y., (1989), the disclosure of which is hereby incorporated by reference in its entirety.) For example, the hCG may be isolated and purified by methods including ammonium sulfate or ethanol precipitation, acid extraction, anion or cation exchange chromatography, phosphocellulose chromatography, hydrophobic interaction chromatography, affinity chromatography, and hydroxyapatite chromatography. Protein refolding steps can be used, as necessary, in completing configuration of the protein. Finally, high performance liquid chromatography can be employed for final purification steps.

In one alternative, a sample suspected of containing human chorionic gonadotropin is subjected to immunoprecipitation or to immunoaffinity purification using an antibody which recognizes human chorionic gonadotropin. Isolated human chorionic gonadotropin then is subjected to gel electrophoresis, such as, for example, sodium dodecyl sulfate-polyacrylamide gel electrophoresis, and transferred to an appropriate membrane, such as a polyvinylidene fluoride (PVDF) membrane. Appropriate bands then are excised from the membrane, and treated with an exoglycosidase or with a combination of an exoglycosidase and a neuraininidase to release the oligosaccharides from the human chorionic gonadotropin. The oligosaccharides then are labeled and analyzed through separation of the oligosaccharides on a gel. The gel then is analyzed for the presence of oligosaccharides with enhanced fucosylation and/or triantennary branching.

In another alternative, two assays are conducted. In the first assay, a carbohydrate probe such as Aleuria aurantia lectin, Galanthus nivalis agglutinin, Sambucis nigra agglutinin, or Phaseolus vulgaris erythroagglutinin is incubated with a predetermined amount of a first portion of a sample suspected of containing human chorionic gonadotropin. After incubation, the sample is contacted with an antibody supported on a solid support. In the preferred embodiment, the supported antibody, which may be a monoclonal or polyclonal antibody, is cleaved by a protease to remove the carbohydrate moiety on the Fc region. In a preferred embodiment the antibody or antibody fragment recognizes an epitope of human chorionic gonadotropin which, if an oligosaccharide that binds the carbohydrate binding agent is present on the hCG, is proximal to such oligosaccharide, whereby binding of the hCG antigen to the supported antibody is inhibited by the binding of the carbohydrate binding agent. If hCG having linked oligosaccharides that bind the carbohydrate binding agent (preferably Aleuria aurantia lectin, Galanthus nivalis agglutinin, Sambucis nigra agglutinin, or Phaseolus vulgaris erythroagglutinin) is present in such sample, such hCG will not be able to bind to the supported antibody due to the previous binding of the carbohydrate binding agent. If there is human chorionic gonadotropin present in the sample which does not include oligosaccharides that bind lectin, such human chorionic gonadotropins will bind to the solid support. Thus, any hCG which binds to the supported antibody is hCG which does not bind the carbohydrate binding agent and thus is less modified by these specific oligosaccharides. Bound hCG then is separated from free hCG. The sample then is contacted with a labeled antibody which recognizes an epitope of human chorionic gonadotropin. Thus, the labeled antibody binds to human chorionic gonadotropin which has been bound by the supported antibody. Bound labeled antibody then is separated from free labeled antibody. The amount of bound, labeled antibody is then determined.

In the second assay, the antibody or antibody fragment reactive to hCG which is supported on a solid support (preferably in an oriented manner) is contacted with a second portion of the sample suspected of containing hCG. The second portion contains an amount of sample identical to that of the first portion. After the sample has contacted the solid support, the labeled antibody to hCG is added, and the amount of bound labeled antibody is determined. If the amounts of bound labeled antibody in the first assay and the second assay are the same, then no hCG which binds the carbohydrate binding agent used is present in the sample. If the amount of bound labeled antibody in the second assay is greater than that in the first assay, then hCG which binds the carbohydrate binding agent used is present in the sample.

Carbohydrate Binding

In a further, preferred embodiment of this invention, the glycosylation of antigens is recognized by a method comprising: providing an antibody having an affinity for an antigen; cleaving said antibody using a protease to yield a cleaved antigen recognition fragment; linking said cleaved antigen recognition fragment to a solid matrix; contacting said cleaved antigen recognition fragment with an antigen, yielding a bound antigen; contacting said bound antigen with a carbohydrate-binding molecule; and recognizing the contacted carbohydrate-binding molecule.

In one embodiment of this invention, the carbohydrate binding agent comprises one or more lectins. Lectins are herein defined as a sugar-binding protein of non-immune origin. The structural information obtained from a large number of mammalian lectins has led to their classification into several families which exhibit a number of variable properties, including ion dependence and solubility (see for example, Drickamer, K., J. Biol. Chem., 1988, 263: 9557; Drickamer, K., Curr. Opin. Struc. Biol., 1993, 3: 393; Drickamer, K., Biochemical Society Transactions, 1993, 21: 456). In addition, certain lectins bind DNA molecules, depending on conditions (see Vieira-Breitwieser O, 20^{th} Annual International Lectin Meeting Abstracts, T25, 2002), therefore the conditions of the assay will vary depending on the lectin used. The lectins that can be used in this application include those derived from the group consisting of animal derived, galactose-binding lectins (termed Galectins); Ca-dependent (C-type) animal lectins including sialyl-LewisX recognizing selectins and mannose-specific collectins; glycosaminoglycan binding annexins; plant-derived lectins including concanavalin A, and ricin, and invertebrate lectins such as tachylectins or Xenopus oocyte lectins. Lectins also include: Arachis Hypogaea Agglutinin (PNA), Bauhinia Purpurea Agglutinin (BPA), Bendeirea Simplicifolia Agglutinin (BSA), Canavalia Ensoformis Agglutinin (CON A), Dolichos Biflorus Agglutini (DBA), Glycine Max (SBA), Lens Culinaris (LcH), Limulus Polyhemus (LPA), Lotus Tetragonolobus (Lotus A), Phaseolus Vulgaris (L-PHA), Phaseolus Limensis (LBA I), Phaseolus Vulgaris (H-PHA), Pisum Sativum (PEA), Phytolacca Americana (Pokeweed), Ricinus Communis (RCA I), Ricinus Comunis (RCA II), Sophora Japonica (SJA), Triticum Vulgaris (WGA), Ulex Europeus (UEA I), Ulex Europaeus (UEA II), Wisteria Floribunda (WFA).

The choice of lectin will dictate the subpopulation of glycoproteins that are analyzed in each iteration. Thus, in preferred embodiments, lectins that are known to recognize carbohydrate moieties that are altered in disease states are used. In further preferred embodiments, the lectins used include 1-6 oligosaccharide binding leukoagglutinin (L-PHA), Concanavalin A (ConA), which has affinity for oligomannosyl saccharides found in N-glycans, galectins including Galectin LEC-6, specific for LacNAc-containing glycans, Aleuria aurantia lectin (AAL) with broad specificity for L-Fuc-containing oligosaccharides, Peanut agglutinin (PNA), which is specific for Gall-3GalNAc), found widely in O-glycans, and Helix pomatia lectin (HPA), which binds N-acetylgalactosamine, Sambucis nigra agglutinin, Galanthus nivalis agglutinin, or Phaseolus vulgaris erythroagglutinin.

In addition to conjugated lectins, other non-lectin sugar binding proteins including sugar-specific enzymes and transport proteins can be used in this method. Mono- or poly-clonal antibodies specific to one or more carbohydrate moieties may be used. These can include antibodies that are commercially available, such as anti-blood Group A Isotypes; anti-blood Group B Isotype; anti-blood Group H Isotypes; anti-Le Isotypes; anti-GM3 Isotypes; anti-E-selectin Isotypes; anti-MUC1 Isotypes; anti-Extended sialyl-Lewis Isotypes anti-Gb3 Isotypes.

In a specific embodiment of this invention, hCG is immobilized and the bound hCG then is contacted with a compound or binding molecule that probes for enhanced fucosylation and/or triantennary branching, or decreased terminal mannose, and most preferably Aleuria aurantia lectin, Galanthus nivalis agglutinin, Sambucis nigra agglutinin, or Phaseolus vulgaris erythroagglutinin.

One embodiment of the invention is specific for hCG modifications which can be used to determine levels of tumoral hCG in a sample. To determine the presence of the oligosaccharides on hCG that bind Aleuria aurantia lectin, Galanthus nivalis agglutinin, Sambucis nigra agglutinin, or Phaseolus vulgaris erythroagglutinin, in one embodiment hCG antigen is isolated from a biological sample derived from a patient and any assay described herein is followed. The biological sample may be derived from serum, plasma, prostatic fluid, semen, urine, spinal fluid, or other suitable biological fluid. The assay of the present invention is particularly applicable for detection and quantitation of hCG antigen in various body fluids, such as serum, pleural efflusions, and urine, of carcinoma patients. In the preferred embodiment, the biological sample is derived from urine. This is particularly important clinically because the assay provides early detection of cancer, as altered hCG expression and glycosylation are associated with certain pre-malignant changes of tissues. Similarly, it is possible to detect increased levels of hCG antigens in carcinoma patients before cancer tissues reaches a critical mass which is detectable by other non-invasive methods. In these cases the assay is particularly valuable for its early diagnosis of cancer.

Recognizing Interaction

The interaction of the antibody fragments of the invention with the antigens and carbohydrate recognition agents can be distinguished by a variety of means. In one embodiment, the antibody fragments are linked to a solid matrix and antigen binding is recognized by a perturbation of that matrix. This can occur through, in one example, surface plasmon resonance (SPR). SPR is a phenomenon which occurs when light is reflected off thin metal films. A fraction of the light energy incident at a sharply defined angle can interact with the delocalized electrons in the metal film (plasmon) thus reducing the reflected light intensity. The precise angle of incidence at which this occurs is determined by a number of factors, preferably the refractive index close to the backside of the metal film, to which target molecules are immobilized and addressed by ligands in a mobile phase running along a flow cell. If binding occurs to the immobilized target the local refractive index changes, leading to a change in SPR angle, which can be monitored in real-time by detecting changes in the intensity of the reflected light, producing a sensorgram. The rates of change of the SPR signal can be analyzed to yield apparent rate constants for the association and dissociation phases of the reaction. The ratio of these values gives the apparent equilibrium constant (apparent affinity). The size of the change in SPR signal is directly proportional to the mass being immobilized and can thus be interpreted crudely in terms of the stoichiometry of the interaction. Signals are easily obtained from sub-microgram quantities of material. It will also be appreciated that the invention also can be practiced using surface plasmon resonance in which binding of the lectin to the carbohydrate moiety of the antigen is detected by variations in the local refractive index.

In a further embodiment of the invention, the carbohydrate binding agent may be conjugated to a detectable marker or label. Examples of such detectable markers or labels include, but are not limited to, fluorescent labels, radioisotopes, such as, for example, isotopes of iodine, cobalt, or tritium, spin labels, chemiluminescent materials, enzymes, absorbing dyes, biotin, or colored particles. After the bound antigen is contacted with the carbohydrate-binding agent, such as a lectin, the free (unbound) binding molecule is separated from the binding molecule which has been bound. The presence of the analyte contacted with the binding molecule, and therefore having linked oligosaccharides that bind to the carbohydrate binding molecule, can be determined by detecting this label. The presence of the label on the bound antigen may be determined by means known to those skilled in the art, such means being dependent upon the type of label employed. The presence of a label indicates that the bound analyte includes linked oligosaccharide, the levels of which can be determined by means known to those skilled in the art, for instance by fluorescence detection or by chemiluminescence.

In accordance with another aspect of the present invention, there is provided a kit or package for detecting hCG linked to an oligosaccharide that binds carbohydrates having enhanced fucosylation and/or triantennary branching, and/or decreased terminal mannose residues, such as Aleuria aurantia lectin, Sambucis nigra agglutinin, Galanthus nivalis agglutinin, or Phaseolus vulgaris erythroagglutinin. The kit includes (a) a solid support upon which is supported a binder, in particular, a cleaved antibody, which recognizes an epitope of human chorionic gonadotropin; and (b) a binding molecule or probe such as Aleuria aurantia lectin, Sambucis nigra agglutinin, Galanthus nivalis agglutinin, or Phaseolus vulgaris erythroagglutinin having a label such as those hereinabove described.

Thus, in accordance with a further aspect of the present invention, there is provided a kit or package for detecting human chorionic gonadotropin linked to an oligosaccharide that binds Aleuria aurantia lectin, Sambucis nigra agglutinin, Galanthus nivalis agglutinin, or Phaseolus vulgaris erythroagglutinin. The kit includes a pair of solid supports. Each support includes a binder, in particular, an antibody, which recognizes an epitope of human chorionic gonadotropin. The epitope is located proximal to an oligosaccharide which binds Aleuria aurantia lectin, Sambucis nigra agglutinin, or Phaseolus vulgaris erythroagglutinin, if such an oligosaccharide is present on the human chorionic gonadotropin. The kit also includes a probe for carbohydrates that have enhanced fucosylation and/or triantennary branching, or decreased terminal mannose, such as Aleuria aurantia lectin, Sambucis nigra agglutinin, Galanthus nivalis agglutinin, or Phaseolus vulgaris erythroagglutinin, and a labeled antibody which recognizes an epitope of human chorionic gonadotropin.

The binding profiles of samples from patients suspected of harboring a disease may be compared with binding profiles from control samples. These profiles can be developed by either comparing control samples in assays conducted at the same time as those conducted on the suspected samples, or may be developed prior to these assays. Thus, a comparison can readily be made to a benchmark set of values in a clinical setting. The binding profiles are the level of binding of specified lectins to arrays of samples contacted with immobilized antibody or antibody fragments described herein. Control samples can include both those that are known to harbor a disease or those that are known to not harbor a disease.

Disease Detection and Treatment

Since the human hCG antigen can also be used as a prognostic marker in various carcinomas, the assay of the present invention is useful as a prognostic test. Therapy can easily be monitored using the test of the present invention. Correlation of hCG antigen expression with tumor burden and tumor progression gives the capability of monitoring the response of the tumor to anti-cancer treatment.

Therefore, a further embodiment of the invention is a method of detecting cancer by contacting a biological sample with a cleaved antibody, preferably an immobilized antibody fragment immunoreactive against hCG as described herein, and detecting the carbohydrate moieties on the hCG derived from the sample by probing the immobilized hCG with one or more lectins, preferably those selected from the group consisting of Aleuria aurantia lectin, Sambucis nigra agglutinin, Phaseolus vulgaris erythroagglutinin, or Galanthus nivalis agglutinin. In a further embodiment of the invention, the same techniques can be applied to detection of pregnancy in biological samples.

In a further embodiment of the present invention, the invention can be utilized to allow more specific targeting of a therapeutic means, such as a chemotherapeutic molecule, to a diseased cell. The preferred embodiment of this invention comprises a therapeutic means linked to cleaved antigen recognition fragments and carbohydrate recognition agents specific for diseased cells. The combination of these recognition agents can allow more specific and effective targeting of the therapeutic means to diseased cells. Suitable therapeutic means can, in non-limiting examples, include: Cisplatin, dacarbazine, cyclophosphamide, mechlorethamine, cytarabine, streptozocin, carboplatin, Mitomycin, carmustine (BCNU), Mitoxantrone, Pentostatin, dactinomycin, Procarbazine, “-rubicins” such as: daunorubicin—idarubicin- doxorubicin, bleomycin, Methotrexate, chlorambucil, Taxol, plicamycin, Etoposide, thioguanine, Fluorouracil, vinblastine, Hydroxyurea, vincristine, Melphalan, Anastrozole, Bicalutamide, Bromocriptine, Buserelin, Busulfan, Capecitabine, Cyproterone acetate, Demeclocycline, Desmopressin, Dexamethasone, Fluoxyinesterone, Flutamide, Folinic Acid, Goserelin acetate, Interferon, Letrozole, Levanisole, Leuprolide acetate, Lomustine, Medroxyprogesterone, Megestrol acetate, Mercaptopurine, Metyrapone, Mitotane, Nilutamide, Nitrogen Mustard, Premarin, Sulfamethoxazole/, Tamoxifen, or any combination thereof. In addition, any therapeutic means of the present invention can be operably linked to the cleaved antigen recognition fragments and carbohydrate recognition agents specific for diseased cells of the invention through a further linking agent.

The term control sample is a biological sample derived from cells under “normal physiological condition” means conditions that are typical inside a living organism or a cell. It will be recognized that the concentration of various salts depends on the organ, organism, cell, or cellular compartment used as a reference. In this application, the term F(ab) and F(ab′) are synonymous. In addition, F(ab)₂ fragments are considered to comprise, in part, two F(ab) fragments.

### EXAMPLES

### Example 1

### Antibody Preparation for ELISA-Based and Biacore Assays

A monoclonal antibody (OEM-148 from OEM Concepts, clone #218-10148) against the β-subunit of hCG was incubated with immobilized pepsin for 16-20 h at 37° C. with shaking in 0.1 M citrate buffer, pH 3.5, with 25 units of pepsin/mg of antibody. The pepsin was inactivated by addition of an equal volume of 0.1 M Tris-HCl, pH 7.5, and then removed with centrifuge filtration over a 0.2 μm filter. Separation of the F(ab)₂ fragment from residual IgG and F_{c} was accomplished with Protein G affinity chromatography. Fragmentation was verified on a 10% polyacrylamide gel in the presence or absence of 2 mM dithiothreitol. Cleavage with pepsin was more effective for the OEM antibody in terms of yielding greater activity compared to cleavage with papain and ficin. For other monoclonal antibodies, however, ficin treatment yielded the greatest activity.

### Example 2

### Controls to Check Lectin Binding to Antibodies and Fragments

Antibodies or F(ab)₂ were immobilized to microplate wells at 2.5 μg/ml for 2 h at 37° C. and then blocked overnight in blocking buffer (1% bovine serum albumin in phosphate-buffered saline (PBS) with 0.2% Tween) at 4° C. The plate was then probed with an array of biotinylated lectins (40 μg/ml) diluted in blocking buffer, followed by streptavidin conjugated to horseradish peroxidase (HRP) diluted in blocking buffer, each for 2 h at 37° C. Biotinylation of antibodies and fragments was accomplished with amine and sulfhydryl coupling; carboxyl and carbohydrate (IgG) coupling is also applicable. Oxidation of HRP substrate 3,3,5,5 tetrarethylbenzidine (TMB) was quantified using a plate reader at 450 nm. Absorbance readings from blank wells incubated without lectins were subtracted from experimental wells.

### Example 3

### ELISA-Based Assay

JAR cell culture and hCG acquisition: JAR choriocarcinoma cells were acquired from ATCC and cultured according to their guidelines. Briefly, cells were grown in 75 mm² flasks in RPMI media supplemented with 4.5 g/L of glucose, 2 mM L-glutamine, 10 mM HEPES, 1 mM sodium pyruvate, 10% fetal bovine serum, and 1× penicillin-streptomycin-Fungizone. After 48 h of standard culture, the medium was replaced with serum-free medium for 48 h before collection in a 50 ml centrifuge tube. Any suspended cells or cellular debris were first removed by low-speed centrifugation. The clarified medium was concentrated 10-fold in a 10 centrifuge filtration device, and the free hCG present was measured with a radioimmunoassay.

Lectin binding on hCG derived from pregnancy and malignancy: The concentration of pregnancy-derived hCG was verified with the RIA. JAR cell culture media and pregnancy urine-derived hCG were diluted to the same concentration of hCG in PBS and incubated on a microwell plate (7.5 ng/well) after coating with the F(ab)₂ as above. Lectin binding was quantified as described (see above) for the antibody. PBS alone was used as a negative control for residual lectin binding to the antibody fragment.

### Example 4

### Biacore Assay

All experiments utilized a CM5 chip, containing four flow channels comprised of a carboxymethyl dextran matrix on a gold substrate. The monoclonal antibody (or protease-derived antibody fragment as described above) was bound through reactive amines to a test channel. The matrix was activated with 50 mM N-hydroxysuccinimide (NHS) and 200 mM ethyl-N (dimethyaminopropyl) carbodimide (EDC) flowing at 5 μl/min for 7 min. The antibody (50 μg/ml in 10 mM sodium acetate buffer, pH 5.0) was passed over the chip at 5 μl/min until 10,000 RU are reached on the instrument (10-15 min). After immobilization, the surface was deactivated with 1 M ethanolamine, pH 8.5 (5 μl/min for 7 min). A deactivated but non-derivitized reference channel served as control for bulk effect, injection noise, baseline drift, and non-specific binding on the matrix. hCG, standard or tumor cell-secreted, was passed over the test channel in running buffer (10 mM HEPES, pH 7.4, 0.15 M NaCl, 2 mM CaCl₂, 2 mM MgCl₂, 0.005% Tween 20), typically at 5 μl/min for 4 min. After hCG binding, lectins (5 μl/min for 2 min) at a saturating concentration of 500 μg/ml were injected onto the chip. Streptavidin can also be coated to the chip via amine coupling, followed by capture of amine-coupled biotinylated antibody fragment.

To regenerate the antibody for testing additional lectins or hCG samples, the surface is pulsed with either 50 mM HCl at 10 μl/min for 1 min or 10 mM glycine, pH 2.0, at 50 μl/min for 12 sec, depending upon the antibody used. Binding of any component is calculated as the difference in the resonance units 10 sec before the injection and 5 sec before the injection is complete.

### Example 5

### Distinguishing Normal and Aberrant Glycosylation on hCG

The ELISA-based and Biacore protocols enable a distinction of normal glycosylation from abnormal glycosylation of hCG using a unique sandwich assay involving hCG protein capture via an antibody or antibody fragment, followed by lectin binding.

The following lectins bound more to hCG derived from human pregnancy urine:
Amaranthus caudatus lectin (specificity for Gal (1-3)GalNac)
Maackia amurensis lectin I (specificity for (NeuAc (2-6))Neuac (2-3) Gal (1-3) GalNac)
Pisum satvium agglutinin (specificity for glucose/mannose)
Galanthus nvalis agglutinin

The following lectins bound to hCG derived from human choriocarcinoma cells more than to hCG from pregnancy urine:
Aleuria aurantia lectin (specificity for fucose)
Phaseolus vulgaris erythroagglutin (specificity for Gal (1-4)GlcNac (1-2)Man)
Sambucis nigra agglutinin

### Example 6

### Cell Culture and hCG Acquisition

Cancerous cells were acquired from ATCC and cultured according to their guidelines. Briefly, cells were grown in 75 mm² flasks in the recommended media supplemented by 1× penicillin-streptomycin-Fungizone. After 48 h of standard culture, the medium was replaced with serum-free medium for 48 h before collection in a 50 ml centrifuge tube. Any suspended cells or cellular debris were first removed with a 5 min spin at 2000 rpm before the clarified media was concentrated roughly 10-fold in a 10 kD centrifuge filtration device. Intact hCG (Coat-a-Count IRMA, Diagnostics Products Corporation) and total β-subunit (RIA-ICN Pharmaceuticals) present were measured in 3 experiments each. Pregnancy-derived hCG was purchased from Sigma, and its concentration was also verified with IRMA and RIA.

### Example 7

### Antibody Cleavage

Monoclonal antibodies which recognize separate epitopes on hCGβ can be used. To serve as a control for non-specific binding and bulk refractive index changes in the biosensor, an anti-fluorescein isothiocyanate (FITC) antibody, chosen for its lack of reactivity with human serum, can be purchased from Sigma. The antibodies must be treated to remove the one site of N-linked glycosylation on each heavy chain or undesired binding will introduce high background on subsequent incubation with lectins. Each MoAb can be enzymatically digested to sever the carbohydrate-containing F_{c} portion.

A first antibody can be incubated with 100 units of immobilized ficin (Sigma)/mg MoAb in 0.1 M citrate buffer, pH 6.0, 5 mM EDTA ([ethylenedinitrilo]tetraacetic acid), and 1 mM cysteine for 2 h at 37° C. with shaking. Similarly, a second antibody can be digested with immobilized papain, pre-activated with 10 mM cysteine for 30 min at 37° C. in 0.1 M acetate buffer, pH 5.5, 3 mM EDTA. The reaction can be centrifuged over a 0.22 μm centrifuge filter (Millipore) to remove the cysteine, and the enzyme-linked beads can be resuspended in cysteine-free acetate buffer. After completion, each reaction volume can be passed over a 0.22 μm filter to remove the agarose-bound enzymes. The antibody fragments can be dialyzed across a 10 kD membrane into PBS. The F(ab′)₂ fragments can be gently reduced to F(ab′) by incubation with 5-10 mM mercaptoethanolamine for 1 h at 37° C., and dialyzed into immobilization buffer (10 mM sodium acetate, pH 4.0-4.5, 5 mM EDTA). Fragmentation and reduction of the antibodies can be verified on a 10% polyacrylamide gel stained with silver.

### Example 8

### Immobilization of MoAbs

F(ab′) fragments can be immobilized to a CM5 chip (Biacore) through their free hinge thiol groups using a maleimide coupling protocol provided by Biacore. Either anti-FITC, a single antibody, or a mixture of antibodies together, can be diluted to a total concentration of 50 μg F(ab′)/ml of immobilization buffer. During immobilization, flow over the chip can be set at 5 μl/min with running buffer (10 mM HEPES (N-[2-hydroxyethyl] piperazine-N′-[2-ethanesulfonic acid]), pH 7.5, 150 mM NaCl, 3 mM EDTA, 0.005% Tween 20). The chip can be activated with 10 μl of 200 mM EDC (1-ethyl-3-[3-dimethylaminopropyl] carbodiimide hydrochloride)/50 mM NHS (N-hydroxysuccinimide), followed by 20 μl of ethylenediamine, pH 8.5. The hetero-bifunctional cross-linking agent sulfo-MBS (40 μl of 50 mM m-maleimidobenzoyl-N-hydroxysulfo-succinimide ester, Pierce Chemical Company) can bridge the MoAb fragments (40 μl) to the chip. Any remaining active sites can be blocked with 50 μl of 100 mM cysteine in 10 mM sodium acetate buffer, pH 4.5. To avoid analyte depletion during experiments to detect avidity effects, antibody channels can be paired with a separate anti-FITC reference lane. In this manner, approximately 10,000 resonance units (RU) of F(ab′) can be affixed to each channel.

### Example 9

### Avidity Effects of Immobilizing Multiple Monoclonals

A concentration series of pregnancy-derived hCG can be separately injected in HBS over the coupled reference/experimental channels and the binding response recorded on surfaces derivitized with one monoclonal antibody (or fragment) or derivitized with multiple monoclonal antibodies (or fragments) that recognize separate epitopes on hCG. The channels can be regenerated to the antibody fragment between hCG injections with a 30s pulse of 10 mM glycine, pH 2.0 at 30 μl/min flow rate. Avidity effects, manifested thorough decreased dissociation of hCG from multiple monoclonals over single antibodies, increase the apparent affinity for hCG up to 8-fold.

### Example 10

### Lectin Binding on hCGβ Derived from Pregnancy and Malignancy

Urine samples from patients with pregnancy, complete hyaditiform mole, choriocarcinoma or germ cell tumors can be diluted into HBS to give a binding response of approximately 150 RU when injected over a F(ab′) surface of approximately 12,000 RU. Flow rates for all injections but the regeneration pulse can be 5 μl/min. The hormone-containing samples can be injected for 5 min, followed by a 4 min injection of Galanthus nivalis agglutinin, Phaseolus vulgaris erythroagglutinin or Sambucis nigra agglutinin (250 μg/ml with 100 μg/ml bovine serum albumin in HBS). The sensor chip can be regenerated between samples with 15 μl of 10 mM glycine, pH 2.0 flowing at 30 μl /min. Deflections on the sensorgrams caused by residual lectin binding to the antibody fragments or injection noise can be removed by subtracting the response of a blank injection of running buffer followed by the lectin from each experimental sensorgram using BIAevaluation software from BlAcore. Similarly, deflections due to mass transfer effects, air bubbles, and other random events can be removed by subtracting the sensorgram simultaneously generated on a non-derivitized reference channel from each experimental sensorgram. All experiments were repeated three times. Galanthus nivalis agglutinin has shown more binding to diseased samples; Phaseolus vulgaris erythroagglutinin has shown more binding to pregnancy samples. This trend has continued when pregnancy and choriocarcinoma samples were diluted over a 16-fold range. A ratio above two of Phaseolus vulgaris erythroagglutinin binding to Galanthus nivalis agglutinin binding can distinguish diseased samples from pregnancy samples.

### Example 11

### Lectin Binding to hCGβ from other Malignant Cell Lines

BeWo choriocarcinoma cells were obtained from the ATCC and grown according to their instructions in 75 mm² flasks. When the cultures reached 50% confluency, the flasks were washed 3 times with PBS, and the media was replaced with serum-free media for 48 h. The conditioned media was collected and concentrated 10-fold over 10 kD centrifuge filters. Hormone levels were measured with a commercial β-radioimmunoassay. Commercial hCG purified from pregnancy urine (Sigma) was diluted in cell media to the same concentration as hCG from the BeWo cells. Both were then diluted into HBS to 1.0 μg/ml. Lectin probing and channel regeneration were as described above. The lectins Phaseolus vulgaris erythroagglutinin and Sambucis nigra agglutinin bound more extensively to choriocarcinoma cell-derived hCG than pregnancy-derived hCG.

### Examples 12-13

Fifty micrograms of IgG can be digested to F(ab′)2 with 1 mg of immobilized ficin (Sigma) in 0.1 M citrate buffer, pH 6.0, 1 mM cysteine, 5 mM EDTA. After digestion, the reaction volume can be passed over a 0.22 μm centrifuge filter to remove the agarose-bound enzyme. The fragmented antibody can be dialyzed across a 10 kD membrane into PBS and gently reduced by incubation with 5 mM mercaptoethanolamine at 37° C. for 1 h. The resulting F(ab′) fragments can be dialyzed across a 10 kD membrane into immobilization buffer (10 mM sodium acetate, pH 4.0). Fragmentation of the antibody can be verified on a 10% polyacrylamide gel stained with silver.

F(ab′) fragment can be immobilized to a CM5 chip (BIAcore) through its free hinge thiol groups using a maleimide coupling protocol provided by BIAcore. Any remaining active sites can be blocked with 50 μl of 100 mM cysteine in 10 mM sodium acetate buffer, pH 4.5. In this manner, approximately 12,000 resonance units (RU) of F(ab′) can be affixed to a channel.

Flow rates for all injections but the regeneration pulse can be 5 μl/min. The hormone-containing samples can be injected for 5 min, followed by a 4 min injection of the lectin (250 μg/ml with 100 μg/ml bovine serum albumin in HBS). The sensor chip can be regenerated between samples with 15 μl of 10 mM glycine, pH 2.0 flowing at 30 μl /min. Deflections on the sensorgrams caused by residual lectin binding to the antibody fragments or injection noise can be removed by subtracting the response of a blank injection of running buffer followed by the lectin from each experimental sensorgram using BIAevaluation software from BlAcore. Similarly, deflections due to mass transfer effects, air bubbles, and other random events were removed by subtracting the sensorgram simultaneously generated on a non-derivitized reference channel from each experimental sensorgram.

### Example 14

### Comparison of Binding Density of Antibody after Cleavage

IgG and F(ab′) can be compared in their capacity to be linked to BIAcore chip surfaces with amine capture. IgG can be digested with 1 mg of immobilized ficin (Sigma)/50 μg IgG in 0.1 M citrate buffer, pH 6.0, 1 mM cysteine, 5 mM EDTA. After digestion, the reaction volume can be passed over a 0.22 μm centrifuge filter to remove the agarose-bound enzyme. The fragmented antibody can be dialyzed across a 10 kD membrane into PBS and gently reduced by incubation with 5 mM mercaptoethanolamine at 37° C. for 1 h. The resulting F(ab′) fragments can be dialyzed across a 10 kD membrane into immobilization buffer (10 mM sodium acetate, pH 4.0). The F(ab′) fragment can be immobilized to a CM5 chip (BIAcore) through its free hinge thiol groups using a maleimide coupling protocol provided by BlAcore. IgG can be immobilized to a BIAcore chip using amine coupling. Passing hCG over these surfaces at 10 ng/ml at 5 μl/min for 5 min leads to a 3-fold greater deflection on the cleaved antibody surface than that on intact IgG. Thus, the cleaved antibody surface has higher sensitivity given the same amount (approximately 10,000 RU) of immobilized capture molecules.

### REFERENCES

Oelschlaeger P. Srikant-Iyer S. Lange S. Schmitt J. Schmnid R. Fluorophor-linked immunosorbent assay: a time- and cost-saving method for the characterization of antibody fragments using a fusion protein of a single-chain antibody fragment and enhanced green fluorescent protein. Analytical Biochemistry. 309(1):27, Oct. 1, 2002.

Ramirez N. Ayala M. Lorenzo D. Palenzuela D. Herrera L. Doreste V. Perez M. Gavilond J V. Oramas P. Expression of a single-chain Fv antibody fragment specific for the hepatitis B surface antigen in transgenic tobacco plants. Transgenic Research. 11(1):61-4, February 2002.

Hock B. Seifert M. Kramer K. Engineering receptors and antibodies for biosensors. Biosensors & Bioelectronics. 17(3):239-49, March 2002.

Foy B D. Killeen G F. Frohn R H. Impoinvil D. Williams A. Beier J C. Characterization of a unique human single-chain antibody isolated by phage-display selection on membrane-bound mosquito midgut antigens. Journal of Immunological Methods. 261(1-2):73-83, Mar. 1, 2002.

Battersby J E. Snedecor B. Chen C. Champion K M. Riddle L. Vanderlaan M. Affinity-reversed-phase liquid chromatography assay to quantitate recombinant antibodies and antibody fragments in fermentation broth. Journal of Chromatography. A. 927(1-2):61-76, Aug. 24, 2001.

Stausbol-Gron B. Jensen K B. Jensen K H. Jensen M O. Clark B F. De novo identification of cell-type specific antibody-antigen pairs by phage display subtraction. Isolation of a human single chain antibody fragment against human keratin 14. European Journal of Biochemistry. 268(10):3099-107, May 2001.

Schmiedl A. Breitling F. Dubel S. Expression of a bispecific dsFv-dsFv′ antibody fragment in Escherichia coli. Protein Engineering. 13(10):725-34, October 2000.

Schmiedl A. Breitling F. Winter C H. Queitsch I. Dubel S. Effects of unpaired cysteines on yield, solubility and activity of different recombinant antibody constructs expressed in E. coli. Journal of Immunological Methods. 242(1-2):101-14, Aug. 28, 2000.

Horn I R. Wittinghofer A. de Bruine A P. Hoogenboom H R. Selection of phage-displayed fab antibodies on the active conformation of ras yields a high affinity conformation-specific antibody preventing the binding of c-Raf kinase to Ras. FEBS Letters. 463(1-2):115-20, Dec. 10, 1999.

Grant S D. Porter A J. Harris W J. Comparative sensitivity of immunoassays for haptens using monomeric and dimeric antibody fragments. Journal of Agricultural & Food Chemistry. 47(1):340-5, January 1999.

Sanchez L. Ayala M. Freyre F. Pedroso I. Bell H. Falcon V. Gavilondo J V. High cytoplasmic expression in E. coli, purification, and in vitro refolding of a single chain Fv antibody fragment against the hepatitis B surface antigen. Journal of Biotechnology. 72(1-2):13-20, Jun. 11, 1999.

Casalvilla R. Duenas M. Ayala M. Cruz S. Cruz L. Buurman W A. Gavilondo J V. A bacterial single-chain Fv antibody fragment that inhibits binding of its parental anti-E-selectin monoclonal antibody to activated human endothelial cells. Journal of Biotechnology. 72(1-2):1-12, Jun. 11, 1999.

Hengerer A. Kosslinger C. Decker J. Hauck S. Queitsch I. Wolf H. Dubel S. Determination of phage antibody affinities to antigen by a microbalance sensor system. Biotechniques. 26(5):956-60, 962, 964, May 1999.

Reinhold U. Liu L. Ludtke-Handjery H C. Heuser C. Hombach A. Wang X. Tilgen W. Ferrone S. Abken H. Specific lysis of melanoma cells by receptor grafted T cells is enhanced by anti-idiotypic monoclonal antibodies directed to the scFv domain of the receptor. Journal of Investigative Dermatology. 112(5):744-50, May 1999.

Piervincenzi R T. Reichert W M. Hellinga H W. Genetic engineering of a single-chain antibody fragment for surface immobilization in an optical biosensor Biosensors & Bioelectronics. 13(3-4):305-12, Mar. 1, 1998.

Gill A. Leatherbarrow R J. Hoare M. Pollard-Knight D V. Lowe P A. Fortune D H. Analysis of kinetic data of antibody-antigen interaction from an optical biosensor by exponential curve fitting. Journal of Biotechnology. 48(1-2):117-27, Jul. 18, 1996.

De Jonge J. Brissinck J. Heirman C. Demanet C. Leo O. Moser M. Thielemans K. Production and characterization of bispecific single-chain antibody fragments. Molecular Immunology. 32(17-18):1405-12, December 1995.

Canaan-Haden L. Ayala M. Fernandez-de-Cossio M E. Pedroso I. Rodes L. Gavilondo J V. Purification and application of a single-chain Fv antibody fragment specific to hepatitis B virus surface antigen. Biotechniques. 19(4):606-8, 610, 612 passim, October 1995.

Graham B M. Porter A J. Harris W J. Cloning, expression and characterization of a single-chain antibody fragment to the herbicide paraquat. Journal of Chemical Technology & Biotechnology. 63(3):279-89, July 1995.

Ayala M. Balint R F. Fernandez-de-Cossio L. Canaan-Haden J W. Larrick J W. Gavilondo J V. Variable region sequence modulates periplasmic export of a single-chain Fv antibody fragment in Escherichia coli. Biotechniques. 18(5):832, 835-8, 840-2, May 1995.

Hobbs S M, Jackson L E, Hoadley J, Interaction of aglycosyl immunoglobulins with the IgG Fc transport receptor from neonatal rat gut: comparison of deglycosylation by tunicamycin treatment and genetic engineering. Molecular Immunology. 29(7-8):949-56, 1992

Chappey O N, Sandouk P, Scherrmann J M. Monoclonal antibodies in hapten immunoassays. Pharm Res. 1992; 9(11):1375-9.

We claim:

1) A method of recognizing an antigen in a sample comprising:
a) providing an antibody having an affinity for an antigen;
b) cleaving said antibody to yield a cleaved antigen recognition fragment;
c) linking said fragment to a solid matrix;
d) contacting a sample and said fragment; and
e) detecting antigen binding to said fragment.

2) The method of claim 1 wherein said sample is urine.

3) The method of claim 1 wherein said sample is blood or a product thereof.

4) The method of claim 1 wherein said antigen is human chorionic gonadotrophin or a peptide fragment thereof.

5) The method of claim 1 wherein said cleaving comprises contacting said antibody with a protease, yielding a cleaved antigen recognition fragment, wherein said protease renders said antigen recognition fragment free of N-linked carbohydrate moieties.

6) The method of claim 1 wherein said cleaving comprises contacting said antibody with a protease, wherein said protease is selected from the group consisting of pepsin, papain, and ficin.

7) The method of claim 1 further comprising separating said cleaved antigen recognition fragment from a cleaved Fc region.

8) The method of claim 1 further comprising comparing said binding to a binding profile in control samples known not to harbor said disease.

9) The method of claim 1 wherein said antigen recognition fragment is a F(ab′) fragment.

10) The method of claim 1 wherein said fragment is linked to said matrix by a disulfide bond.

11) The method of claim 1 wherein said fragment is linked to said matrix by a free carboxy-terminal amino acid through an amide linkage.

12) A method of recognizing glycosylation of an antigen in a sample comprising:
a) providing an antigen recognition fragment linked to a solid matrix;
b) contacting said fragment with an antigen, yielding a bound antigen;
c) contacting said bound antigen with a carbohydrate-binding molecule; and
d) detecting binding of said carbohydrate-binding molecule and said bound antigen.

13) The method of claim 12 wherein Fe is substantially absent from said antigen recognition fragment.

14) The method of claim 12 wherein said sample is urine.

15) The method of claim 12 wherein said sample is blood or a product thereof.

16) The method of claim 12 wherein said antigen is human chorionic gonadotrophin or a peptide fragment thereof.

17) The method of claim 12 further comprising:
a) providing an antibody having an affinity for an antigen;
b) cleaving said antibody to yield a cleaved antigen recognition fragment; and
c) separating said cleaved antigen recognition fragment from a cleaved Fe region before contacting said, antigen with said carbohydrate-binding molecule.

18) The method of claim 12 wherein said antigen recognition fragment is a F(ab′) fragment.

19) The method of claim 12 further comprising comparing said binding to a binding profile in control samples known not to harbor said disease.

20) The method of claim 12 wherein said sample is tested for pregnancy before contacting with said fragment.

21) The method of claim 12 further comprising:
a) providing an antibody; and
b) contacting said antibody with a protease, yielding said antigen recognition fragment;
c) wherein said protease renders said antigen recognition fragment free of N-linked carbohydrate moieties.

22) The method of claim 12 further comprising:
a) providing an antibody; and
b) contacting said antibody with a protease, yielding said antigen recognition fragment;
c) wherein said protease is selected from the group consisting of pepsin, papain, and ficin.

23) The method of claim 12 wherein said fragment is substantially devoid of sugars.

24) The method of claim 12 wherein said fragment is a recombinant antibody fragment.

25) The method of claim 12 wherein said fragment is linked to said matrix by a disulfide bond.

26) The method of claim 12 wherein said fragment is linked to said matrix by a free carboxy-terminal amino acid through an amide linkage.

27) The method of claim 12 wherein said carbohydrate-binding molecule is a lectin.

28) The method of claim 12 wherein said carbohydrate binding molecule is specific for triantennary branching.

29) The method of claim 12 wherein said carbohydrate binding molecule is specific for fucose linked (α-1,6) to N-acetylglucosamine or to fucose linked (α-1,3) to N-acetyllactosamine related structures.

30) The method of claim 12 wherein said carbohydrate binding molecule is selected from the group consisting of Galanthus nivalis agglutinin, Phaseolus vulgaris erythroagglutinin, Sambucis nigra agglutinin, Aleuria aurantia lectin, Amaranthus caudatus lectin, Maackia amurensis lectin, and Pisum satvium agglutinin.

31) The method of claim 12 wherein said detecting comprises recognizing a change in electrochemical or fluorometric properties of the solid matrix, as measured by surface plasmon resonance.

32) The method of claim 12 further comprising: comparing the level of said binding to a binding profile in control samples known not to harbor said disease.

33) A method of detecting a tumor in an animal comprising:
a) providing an antibody or fragment thereof having affinity for human chorionic gonadotrophin (hCG);
b) providing a carbohydrate binding molecule that recognizes enhanced fucosylation and/or triantennary branching and/or decreased terminal mannose residues;
c) contacting said antibody or fragment thereof with a sample;
d) contacting said carbohydrate-binding molecule with said sample before, after, or simultaneously with step (c); and
e) detecting binding between said hCG and said carbohydrate-binding molecule;
f) wherein the antibody or fragment thereof or carbohydrate binding molecule is bound to a solid matrix.

34) The method of claim 33 wherein said sample is urine.

35) The method of claim 33 wherein said sample is blood or a product thereof.

36) The method of claim 33 further comprising comparing the level of said binding to a binding profile in control samples known not to harbor said tumor.

37) The method of claim 33 further comprising contacting said antibody with a protease, yielding an antigen recognition fragment wherein said protease renders said antigen recognition fragment free of N-linked carbohydrate moieties.

38) The method of claim 33 further comprising contacting said antibody with a protease, yielding an antigen recognition fragment wherein said protease is selected from the group consisting of pepsin, papain, and ficin.

39) The method of claim 33 further comprising contacting said antibody with a protease, yielding an antigen recognition fragment and separating said antigen recognition fragment from an Fc region.

40) The method of claim 33 wherein said antibody fragment is a F(ab′) fragment.

41) The method of claim 33 wherein said antibody is substantially devoid of sugars.

42) The method of claim 33 wherein said antibody fragment is a recombinant antibody fragment.

43) The method of claim 33 wherein said antibody is linked to said matrix by a disulfide bond.

44) The method of claim 33 wherein said fragment is linked to said matrix by a free carboxy-terminal amino acid through an amide linkage.

45) The method of claim 33 wherein said carbohydrate-binding molecule is a lectin.

46) The method of claim 33 wherein said carbohydrate binding molecule is specific for tri-antennary branching.

47) The method of claim 33 wherein said carbohydrate binding molecule is specific for fucose linked (α-1,6) to N-acetylglucosamine or to fucose linked (α-1,3) to N-acetyllactosamine related structures.

48) The method of claim 33 wherein said carbohydrate binding molecule is selected from the group consisting of Galanthus nivalis agglutinin, Phaseolus vulgaris erythroagglutinin, Sambucis nigra agglutinin, Aleuria aurantia lectin, Amaranthus caudatus lectin, Maackia amurensis lectin, and Pisum satvium agglutinin.

49) The method of claim 33 wherein said detecting comprises recognizing a change in electrochemical or fluorometric properties of the solid matrix, as measured by surface plasmon resonance.

50) The method of claim 33 further comprising: comparing the level of said binding to a binding profile in control samples known not to harbor said tumor.

51) The method of claim 33 wherein said carbohydrate molecule is Phaseolus vulgaris erythroagglutinin.

52) A method of distinguishing glycosylation of human chorinic gonadotropin antigen in a pregnant female from glycosylation of human chorinic gonadotropin antigen in a female harboring an hCG-secreting cancer comprising:
a) providing an antibody or fragment thereof having affinity for human chorionic gonadotrophin (hCG);
b) providing a carbohydrate binding molecule that recognizes enhanced fucosylation and/or triantennary branching and/or decreased terminal mannose residues;
c) contacting said antibody or fragment thereof with a sample comprising hCG;
d) contacting said carbohydrate-binding molecule with said sample before, after, or simultaneously with step (c);
e) detecting binding between said hCG and said carbohydrate-binding molecule;
f) wherein the antibody or fragment thereof or carbohydrate binding molecule is bound to a solid matrix; and
g) wherein said sample is derived from a female suspected of being pregnant or previously suspected of being pregnant.

53) The method of claim 52 wherein said sample is urine.

54) The method of claim 52 wherein said sample is blood or a product thereof.

55) The method of claim 52 further comprising comparing said binding to a binding profile in control samples known not to harbor cancer secreted hCG.

56) The method of claim 52 wherein said antibody is a F(ab′) fragment.

57) The method of claim 52 wherein said antibody is a recombinant antibody fragment.

58) The method of claim 52 wherein said antibody is linked to said matrix by a disulfide bond.

59) The method of claim 52 wherein said fragment is linked to said matrix by a free carboxy-terminal amino acid through an amide linkage.

60) The method of claim 52 wherein said carbohydrate-binding molecule is a lectin.

61) The method of claim 52 wherein said carbohydrate binding molecule is specific for triantennary branching.

62) The method of claim 52 wherein said carbohydrate binding molecule is specific for fucose linked (α-1,6) to N-acetylglucosamine or to fucose linked (α-1,3) to N-acetyllactosamine related structures.

63) The method of claim 52 wherein said carbohydrate binding molecule is selected from the group consisting of Galanthus nivalis agglutinin, Phaseolus vulgaris erythroagglutinin, Sambucis nigra agglutinin, Aleuria aurantia lectin, Amaranthus caudatus lectin, Maackia amurensis lectin, and Pisum satvium agglutinin.

64) The method of claim 52 wherein said detecting comprises recognizing a change in electrochemical or fluorometric properties of the solid matrix, as measured by surface plasmon resonance.

65) The method of claim 52 wherein said carbohydrate molecule is Phaseolus vulgaris erythroagglutinin.

66) The method of claim 52 further comprising, before step (a), measuring total hCG or fragment thereof in a biological sample from said female and assessing pregnancy.

67) A therapeutic device comprising:
a) a therapeutic means;
b) on or more cleaved antibody fragments attached to said therapeutic means, optionally by a thiol linkage; and
c) one or more lectin molecules attached to said therapeutic means;
d) wherein said lectin molecules and said antibody fragments are specific for a glycoprotein found in samples from diseased animals.

68) A diagnostic kit comprising:
a) a targeting agent for hCG; and
b) a carbohydrate probe specific for enhanced fucosylation and/or triantennary branching and/or decreased terminal mannose.

69) A kit for detecting human chorionic gonadotropin linked to an oligosaccharide that binds Aleuria aurantia lectin, Sambucis nigra agglutinin, Galanthus nivalis agglutinin, or Phaseolus vulgaris erythroagglutinin, comprising:
a) an antibody which recognizes an epitope of human chorionic gonadotropin;
b) a solid support to which said antibody is bound; and
c) Aleuria aurantia lectin, Sambucis nigra agglutinin, Galanthus nivalis agglutinin, and/or Phaseolus vulgaris erythroagglutinin.
